(19) United States
(12) Patent Application Publication
(10) Pub. No.: US 2020/0281925 A1
(43) Pub. Date: Sep. 10, 2020

(54) Dose and regimen for an hdm2-p53 interaction inhibitor in hematological tumors

(71) Applicant: Novartis AG, Basel (CH)

(72) Inventors: Stephane FERRETTI, Huningue (FR); Nesol GURREIO, Basel (CH); Senastien JEAY, Niffer (FR); Astrid JULLION, Sierntz (FR); Christophe MEILLE, Sierentz (FR); Jens WUERTHNER, Steinen (FR)

(21) Appl. No.: 16/494,927

(22) PCT Filed: Mar. 29, 2018
(86) PCT No.: PCT/IB2018/052187

§ 371 Date: Sep. 17, 2019

Related U.S. Application Data
(60) Provisional application No. 62/479,397, filed on Mar. 31, 2017.

(51) Int. Cl. A61K 31/506; A61K 45/06; A61K 31/553; A61K 31/706; A61K 31/7068; A61P 35/00

(52) U.S. Cl. CPC A61K 31/506; A61K 45/06; A61P 35/00; A61K 31/706; A61K 31/7068; A61K 31/553

(57) ABSTRACT

The present invention relates to the HDM2-p53 interaction inhibitors (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (HDM201), or a pharmaceutically acceptable non-covalent derivative thereof, for use in the treatment of patients with hematological tumors, wherein the drug is administered by an extended low dose dosing regimen.

### FIELD OF THE INVENTION

The present invention relates the HDM2-p53 interaction inhibitor (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (HDM201) for use in the treatment of hematological tumors, wherein the drug is administered following an extended low dose regimen.

### BACKGROUND OF THE INVENTION

p53 is induced and activated by a number of potentially tumorigenic processes—including aberrant growth signals, DNA damage, ultraviolet light, and protein kinase inhibitors (Millard M, et al. Curr Pharm Design 2011; 17:536-559)—and regulates genes controlling cell growth arrest, DNA repair, apoptosis, and angiogenesis (Bullock A N & Fersht A R. Nat Rev Cancer 2001; 1:68-76; Vogelstein B, et al. Nature Education 2010; 3(9):6).

Human Double Minute-2 (HDM2) is one of the most important regulators of p53. It binds directly to p53, inhibiting its transactivation, and subsequently directing it towards cytoplasmic degradation (Zhang Y, at al. Nucleic Acids Res 2010; 38:6544-6554).

p53 is one of the most frequently inactivated proteins in human cancer, either through direct mutation of the TP53 gene (found in approximately 50% of all human cancers) (Vogelstein, B et al. Nature 2000; 408:307-310) or via suppressive mechanisms such as overexpression of HDM2 (Zhao Y, et al. BioDiscovery 2013; 8:4).

Potent and selective inhibitors of the HDM2-p53 interaction (also referred to as HDM2 inhibitors or MDM2 inhibitors), e.g. NVP-HDM201, have been shown to restore p53 function in preclinical cell and in vivo models (Holzer P, et al. Poster presented at AACR 2016, Abstract #4855).

Different dosing regimens were described for HDM2 inhibitors and tested in clinical studies. E.g. US2013/0245089 discloses a method of treating a patient suffering from cancer by administering to the patient 4-{[(2R,3S,4R,5S)-4-(4-Chloro-2-fluoro-phenyl)-3-(3-chloro-2-fluoro-phenyl)-4-cyano-5-(2, 2-dimethyl-propyl)-pyrrolidine-2-carbonyl]-amino}-3-methoxy-benzoic acid in an amount of from about 800 to about 3000 mg/day for an administration period of up to about 7 days, on days 1-7, of a 28 days treatment cycle, followed by a rest period of from about 21 to about 23 days.

A paper in Clinical Cancer Research by B. Higgins et al. (May 2014) disclosed a 28-day cycle schedule, where RG7388 is administered once weekly three times followed by 13 days of rest (28 days cycle schedule), or where the drug is administered for 5 consecutive days of a 28 days schedule.

Further dosing regimens for HDM2 inhibitors are disclosed in WO 2015/198266.

The HDM2 inhibitor HDM201, i.e. (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, and methods how to prepare it were disclosed for example in WO2013/111105.

### SUMMARY OF THE INVENTION

One of the objectives in the development of an HDM2 inhibitor drug is to find a dosing regimen which allows the administration of a dose which ensures efficacy but at the same time reduces the risk of the occurrence of adverse events.

It has been surprisingly found that one type of dosing regimen is particularly useful for the treatment of hematological tumors with the HDM2 inhibitor HDM201.

Specifically, the present invention provides the following aspects, advantageous features and specific embodiments, respectively alone or in combination, as listed in the following items:
1. The HDM2-p53 interaction inhibitor drug (S)-5-(5-Chloro-1-methyl-2-oxo-1,2-dihydro-pyridin-3-y)-6-(4-chloro-phenyl)-2-(2,4-dimethoxy-pyrimidin-5-y)-1-isopropyl-5,6-dihydro-1H-pyrrolo[3,4-d]imidazol-4-one (HDM201) or a pharmaceutically acceptable non-covalent derivative (including salt, solvate, hydrate, complex, co-crystal) thereof
   for use in the treatment of hematological tumors,
   wherein the drug is administered on each of the first 6 to 8 days of a 28 days treatment cycle,
   wherein the treatment is composed of at least two 28 days treatment cycles, and
   wherein the daily drug dose is from 40 mg to 90 mg.
2. The HDM2-p53 interaction inhibitor drug HDM201, or non-covalent derivative thereof, for use in the treatment of hematological tumors according to item 1, wherein the daily drug dose is from 40 mg to 60 mg.
3. The HDM2-p53 interaction inhibitor drug HDM201, or non-covalent derivative thereof, for use in the treatment of hematological tumors according to item 1, wherein the daily drug dose is from 40 mg to 50 mg.
4. The HDM2-p53 interaction inhibitor drug HDM201, or non-covalent derivative thereof, for use in the treatment of hematological tumors according to item 1, wherein daily drug dose is 45 mg.
5. The HDM2-p53 interaction inhibitor drug HDM201, or non-covalent derivative thereof, for use in the treatment of hematological tumors according to item 1, wherein the drug is administered once daily on each of the first 7 days (first week) of a 28 days (4 weeks) treatment cycle and the daily drug dose is 45 mg.
6. The HDM2-p53 interaction inhibitor drug HDM201, or non-covalent derivative thereof, for use in the treatment of hematological tumors according to any one of items 1-5, wherein the drug is present as co-crystal, preferably present as succinic acid co-crystal.
7. The HDM2-p53 interaction inhibitor drug HDM201, or non-covalent derivative thereof, for use in the treatment of hematological tumors according to any one of items 1-5, wherein the drug is present as solvate, preferably present as hydrate.
8. The HDM2-p53 interaction inhibitor drug HDM201, or non-covalent derivative thereof, for use in the treatment of hematological tumors according to any one of items 1-5, wherein the drug is present as non-covalent derivative, preferably present as non-covalent derivative comprising succinic acid or water, more preferably present as non-covalent derivative comprising succinic acid.
9. The HDM2-p53 interaction inhibitor drug HDM201, or non-covalent derivative thereof, for use in the treatment of hematological tumors according to any one of items 1-8, wherein the hematological tumor is a leukemia.
10. The HDM2-p53 interaction inhibitor drug HDM201, or non-covalent derivative thereof, for use in the treatment of hematological tumors according to any one of items 1-9, wherein the hematological tumor is selected from acute myeloid leukemia (AML), myelodysplastic syndrome (MDS), and acute lymphoblastic leukemia (ALL).
11. The HDM2-p53 interaction inhibitor drug HDM201, or non-covalent derivative thereof, for use in the treatment of hematological tumors according to any one of items 1-10, wherein the hematological tumor is a TP53 wild-type hematological tumor.
12. The HDM2-p53 interaction inhibitor drug HDM201, or non-covalent derivative thereof, for use in the treatment of hematological tumors according to any one of items 1-11, wherein the hematological tumor is a relapsed/refractory hematological tumor.
13. The HDM2-p53 interaction inhibitor drug HDM201, or non-covalent derivative thereof, for use in the treatment of hematological tumors according to any one of items 1-8, wherein the hematological tumor is a relapsed/refractory TP53 wild-type hematological tumor selected from acute myeloid leukemia (AML), myelodysplastic syndrome (MDS), and acute lymphoblastic leukemia (ALL).

The following items are particularly preferred embodiments of the present invention:
14. The HDM2-p53 interaction inhibitor drug (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (HDM201) as succinic acid co-crystal
   for use in the treatment of relapsed/refractory TP53 wild-type hematological tumors selected from acute myeloid leukemia (AML), myelodysplastic syndrome (MDS), and acute lymphoblastic leukemia (ALL),
   wherein the drug is administered once daily on each of the first 7 days of a 28 days treatment cycle,
   wherein the treatment is composed of at least two 28 days treatment cycles, and
   wherein the daily drug dose is 45 mg.
15. The HDM2-p53 interaction inhibitor drug (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (HDM201) as succinic acid co-crystal
   for use in the treatment of relapsed/refractory TP53 wild-type acute myeloid leukemia (AML),
   wherein the drug is administered once daily on each of the first 7 days of a 28 days treatment cycle,
   wherein the treatment is composed of at least two 28 days treatment cycles, and wherein the daily drug dose is 45 mg.

The dosing regimens of the present invention as described above provide a highly favorable therapeutic index, low incidence of grade 3/4 thrombocytopenia while achieving therapeutically relevant exposures, p53 pathway activation (GDF-15 upregulation), and clinical activity.

### BRIEF DESCRIPTION OF THE DRAWINGS

In the following the present invention is described in detail with reference to accompanying figures in which:

FIG. 1 illustrates the best percentage change in blast percentage in bone marrow (BM) aspirate in AML patients (patients with available bone marrow aspirate).

*Ongoing treatment; # best percentage change is ≥100; TF: treatment failure; CR: complete response; CRi: morphologic CR with incomplete blood count recovery. Daily doses: Regimen 1A: 250, 350 or 400 mg. Regimen 1B: 150 mg. Regimen 2A: 20, 30 mg. Regimen 2C: 45 mg.

FIG. 2 shows the individual average concentration during first treatment cycle versus dose per regimen for patients with hematological tumors.

Line at 120 ng/mL=95% tumor regression from human SJSA-1 xenograft rat. Line at 41 ng/mL=Average concentration for tumor stasis derived from TGI PK/PD modelling in human SJSA-1 (osteosarcoma) xenograft rat. Line at 19 ng/mL—Average concentration for tumor stasis derived from TGI PK/PD modelling in human HSAX2655 (liposarcoma) PDX rat. Calculation of average dose level (mg/day):

| | Daily | No. of | Total dose | Cycle | Average |
|---|---|---|---|---|---|
| | dose | administration | per cycle | duration | dose |
| Regimen | (mg) | days | (mg) | (days) | (mg/day) |
| 1A | 250 | 1 | 250 | 21 | 11.9 |
| | 350 | 1 | 350 | 21 | 16.7 |
| | 400 | 1 | 400 | 21 | 19 |
| 1B | 150 | 2 | 300 | 28 | 10.7 |
| 2A | 20 | 14 | 280 | 28 | 10 |
| | 30 | 14 | 420 | 28 | 15 |
| 2C | 45 | 7 | 315 | 28 | 11.3 |

FIGS. 3-5 illustrates the best percentage change in blast percentage in bone marrow (BM) aspirate in AML patients (cut-off date 15 Jan. 2018).

*Ongoing treatment; # best percentage change is ≥100; TF: treatment failure; CR: complete response; CRi: morphologic CR with incomplete blood count recovery.

Daily doses: FIG. 3: Regimen 1A at 250 mg. FIG. 4: Regimen 1B at 120 mg. FIG. 5: Regimen 2C at 45 mg.

### DETAILED DESCRIPTION OF THE INVENTION

Herein after, the present invention is described in further detail and is exemplified.

In one aspect the invention provides:
The HDM2-p53 interaction inhibitor drug (S)-5-(5-Chloro-1-methyl-2-oxo-1,2-dihydro-pyridin-3-y)-6-(4-chloro-phenyl)-2-(2,4-dimethoxy-pyrimidin-5-y)-1-isopropyl-5,6-dihydro-1H-pyrrolo[3,4-d]imidazol-4-one (HDM201) or a pharmaceutically acceptable non-covalent derivative (including salt, solvate, hydrate, complex, co-crystal) thereof
   for use in the treatment of hematological tumors,
   wherein the drug is administered on each of the first 6 to 8 days, preferably 7 days, of a 28 days treatment cycle,
   wherein the treatment is composed of at least two 28 days treatment cycles, and
wherein the daily drug dose is from 40 mg to 90 mg, preferably 45 mg.

The term “HDM2-p53 interaction inhibitor” or in short “HDM2 inhibitor” is also referred to as “HDM2i”, “Hdm2i”, “MDM2 inhibitor”, “MDM2i”, “Mdm2i”, denotes herein any compound inhibiting the HDM-2/p53 or HDM-4/p53 interaction with an IC₅₀ of less than 10 μM, preferably less than 1 μM, preferably in the range of nM, measured by a Time Resolved Fluorescence Energy Transfer (TR-FRET) Assay. The inhibition of p53-Hdm2 and p53-Hdm4 interactions is measured by time resolved fluorescence energy transfer (TR-FRET). Fluorescence energy transfer (or Foerster resonance energy transfer) describes an energy transfer between donor and acceptor 5 fluorescent molecules. For this assay, MDM2 protein (amino acids 2-188) and MDM4 protein (amino acids 2-185), tagged with a C-terminal Biotin moiety, are used in combination with a Europium labeled streptavidin (Perkin Elmer, Inc., Waltham, Mass., USA) serving as the donor fluorophore. The p53 derived, Cy5 labeled peptide Cy5-TFSDLWKLL (p53 aa18-26) is the energy acceptor. Upon excitation of the donor 10 molecule at 340 nm, binding interaction between MDM2 or MDM4 and the p53 peptide induces energy transfer and enhanced response at the acceptor emission wavelength at 665 nm. Disruption of the formation of the p53-MDM2 or p53-MDM4 complex due to an inhibitor molecule binding to the p53 binding site of MDM2 or MDM4 results in increased donor emission at 615 nm. The ratiometric FRET assay readout is calculated from the 15 raw data of the two distinct fluorescence signals measured in time resolved mode (countrate 665 nm/countrate 615 nm×1000). The assay can be performed according to the following procedure: The test is performed in white 1536w microtiterplates (Greiner Bio-One GmbH, Frickenhausen, Germany) in a total volume of 3.1 μl by combining 100 nl of compounds diluted in 90% DMSO/10% H2O (3.2% final DMSO concentration) with 2 μl Europium 20 labeled streptavidin (final concentration 2.5 nM) in reaction buffer (PBS, 125 mM NaCl, 0.001% Novexin (consists of carbohydrate polymers (Novexin polymers), designed to increase the solubility and stability of proteins; Novexin Ltd., ambridgeshire, United Kingdom), Gelatin 0.01%, 0.2% Pluronic (block copolymer from ethylenoxide and propyleneoxide, BASF, Ludwigshafen, Germany), 1 mM DTT), followed by the addition of 0.5 μl MDM2-Bio or MDM4-Bio diluted in assay buffer (final concentration 10 nM). Allow the solution to pre-incubate for 15 minutes at room temperature, followed by addition of 0.5 μl Cy5-p53 peptide in assay buffer (final concentration 20 nM). Incubate at room temperature for 10 minutes prior to reading the plate. For measurement of samples, an Analyst GT multimode microplate reader (Molecular Devices) with the following settings 30 is used: Dichroic mirror 380 nm, Excitation 330 nm, Emission Donor 615 nm and Emission Acceptor 665 nm. IC50 values are calculated by curve fitting using XLfit. If not specified, reagents are purchased from Sigma Chemical Co, St. Louis, Mo., USA.

The HDM2 inhibitor in accordance with this invention is HDM201, i.e. (S)-5-(5-Chloro-1-methyl-2-oxo-1,2-dihydro-pyridin-3-yl)-6-(4-chloro-phenyl)-2-(2,4-dimethoxy-pyrmidin-5-yl)-1-isopropyl-5,6-dihydro-1H-pyrrolo[3,4-d]imidazol-4-one.

HDM201 may be present as free molecule or in any other non-covalent derivative, including salt, solvate, hydrate, complex, co-crystal or mixtures thereof. HDM201 may be present as acid derivative. The acid derivative may be a salt formed of HDM201 with the acid, or a HDM201 acid complex, or as HDM201 acid co-crystal. Preferably HDM201 is present as co-crystal. Preferably the acid is succinic acid. Most preferably, HDM201 is present as succinic acid co-crystal. Non-covalent derivatives of HDM201 are described in WO2013/111105.

When referring to a dose amount of HDM201 herein, e.g. in mg (milligram), it is meant to be the amount of HDM201 as free base, in contrast to the salt, solvate, complex, or co-crystal.

The term “hematological tumor” refers herein to a cancer that begins in blood-forming tissue, such as the bone marrow, or in the cells of the immune system. Examples of hematological tumors are leukemia, lymphoma, and multiple myeloma. They are also often referred to as blood cancer.

Preferred hematological tumors of the present invention are leukemias. More preferably, the hematological tumors are selected from acute myeloid leukemia (AML), myelodysplastic syndrome (MDS), and acute lymphoblastic leukemia (ALL). Even more preferably, the hematological tumors are an acute leukemia, preferably selected from acute myeloid leukemia (AML), and acute lymphoblastic leukemia (ALL). Even more preferably, the hematological tumor is AML.

Particularly preferred hematological tumors of the present invention are 1P53 wild-type hematological tumor. More preferably, the TP53 wild-type hematological tumors of the present invention are TP53 wild-type leukemias. Even more preferably, the TP53 wild-type hematological tumors are selected from TP53 wild-type acute myeloid leukemia (AML), TP53 wild-type myelodysplastic syndrome (MDS), and TP53 wild-type acute lymphoblastic leukemia (ALL). Even more preferably, the TP53 wild-type hematological tumors are an TP53 wild-type acute leukemia, preferably selected from TP53 wild-type acute myeloid leukemia (AML), and TP53 wild-type acute lymphoblastic leukemia (ALL). Even more preferably, the TP53 wild-type hematological tumor is TP53 wild-type AML.

According to the present invention the drug HDM201 is administered on each of the first 6 to 8 days of a 28 days treatment cycle, preferably on the first seven days (first week) of a 28 days (4 weeks) treatment cycle.

“On the first seven days of a 28 days treatment cycle” means that HDM is administered to the patient on day 1 (d1), d2, d3, d4, d5, d6 and d7 followed by a drug-administration-free period (also referred to as drug holiday period or rest period) from day 8 until day 28. On day 29 the next treatment cycle starts which will be the d1 of this next treatment cycle.

This dosing regimen is also referred to as “1 week on/3 weeks off” or “qd for first week of a 4 week cycle”.

Preferably, the drug is administered at approximately the same time each administration day (i.e. d1-d7 of a 28 days cycle). Preferably, the drug is administered once daily (qd) on each administration day. More preferably, the drug is administered in the morning.

Preferably, the drug is administered in the fasted state, i.e. at least 1 hour before or 2 hours after a meal.

Preferably the drug is taken with a glass of water and without chewing the capsules or tablet.

If the patient is assigned to a dose level where multiple capsules/tablets are to be taken, the capsules/tablets should be taken consecutively, within as short an interval as possible, e.g. within 5 min.

Preferably, the drug administration is done by oral delivery, i.e. oral administration, per oral (p.o.).

Preferably the drug is provided in the form of an oral dosage form, more preferably in the form of a solid oral dosage form, e.g. a capsule or a tablet.

When dose ranges are given herein, e.g. “the daily drug dose is from 40 mg to 90 mg”, any full mg number of the endpoints and in the between those endpoint shall be meant to be disclosed herewith, e.g. 40 mg, 41 mg, 42 mg, 43 mg, 44 mg, 45 mg, 46 mg, 47 mg, . . . 88 mg, 89 mg, 90 mg.

As a further aspect of the present invention there is provided:

A combination of the HDM2-p53 interaction inhibitor drug (S)-5-(5-Chloro-1-methyl-2-oxo-1,2-dihydro-pyridin-3-yl)-6-(4-chloro-phenyl)-2-(2,4-dimethoxy-pyrimidin-5-y)-1-isopropyl-5,6-dihydro-1H-pyrrolo[3,4-d]imidazol-4-one (HDM201) or a pharmaceutically acceptable non-covalent derivative (including salt, solvate, hydrate, complex, co-crystal) thereof with one or more other therapeutically active agents for use in the treatment of hematological tumors, wherein the HDM2-p53 interaction inhibitor drug is administered on each of the first 6 to 8 days of a 28 days treatment cycle,

wherein the treatment is composed of at least two 28 days treatment cycles, and

wherein the daily drug dose is from 40 mg to 90 mg.

The other active agent may be dosed on the same day(s) as HDM201 or on days on which no HDM201 dose is administered.

The other therapeutically active agent is preferably an anti-cancer agent, more preferably said anti-cancer agent may be selected from:

FLT3 inhibitors (e.g. gilterinib, quizartinib, midostaurin),

BCL2 inhibitors (e.g. navitoclax, venetoclax),

other HDM2 inhibitors (e.g. idasanutlin, AMG232, DS-3032B, ALRN6924/ATSP7041),

hypomethylating agents (HMA) (e.g. Vidaza [azacytidine, 5-azacytidine], Dacogen [decitabine], guadecitabine),

anthracyclines (e.g. idarubicin, daunorubicin, doxorubicin, epirubicin);

anti-CD33 antibodies (e.g. Mylotarg [gemtuzumab], vadastuximab)

and other agents (e.g. AraC [cytarabine, aracytine]).

Preferably, the other therapeutical active agent is selected from midostaurin, azacytidine, cytarabine. Preferred combinations are HDM201 with midostaurin, HDM201 with cytarabine, HDM201 with azacytidine.

### EXAMPLES

### Example 1: Clinical Performance of HDM201 Administered in Dosing Regimen 2C

This example provides a summary of the clinical data of the phase 1 trial CHDM201X2101 (data cut-off date of 7 Dec. 2016) that demonstrates that the 45 mg single agent HDM201 daily dose with the extended low dose regimen “2C”, i.e. 1 week on/3 weeks off regimen, for the for patients with hematological tumors treated according to the (HDM201 given 1 week on/3 weeks off) is the most efficient and safest dose/regimen compared to other extended low dose regimens or other intermittent high dose regimens. That the 45 mg HMD201 dosing regimen 2C if the most efficient dosing regimen has been confirmed by the efficacy data at the cut-off date of 15 Jan. 2018.

Herein, data are disclosed from this multicenter, open-label, first-in-human Phase I study of HDM201 in patients with advanced TP53 wild-type (WI) leukemias.

The clinical study design allowed parallel exploration of the safety, tolerability, and clinical activity (efficacy) of two broad dosing strategies for HDM201 during dose escalation: intermittent high dose regimens (Regimen 1A and 1B) and extended low dose regimens (Regimen 2A and 2C). Table 1 summarizes the dosing regimens in each category that were evaluated in patients with hematologic tumors.

TABLE 1
HDM201 Dosing regimens and dose levels
evaluated in hematologic malignancies

| | | | | Total |
|---|---|---|---|---|
| | | Dose levels (number | | number of |
| | Dosing Regimen | of patients) | | patients |
| Intermittent | 1A (d 1 Q3W) | 250 mg | (3) | 16 |
| high dose | | 350 mg | (4) | |
| regimens | | 400 mg | (8AML; 1ALL) | |
| | 1B (d 1, d 8 | 150 mg | (6) | 6 |
| | Q4W) | | | |
| Extended | 2A (2 week on/2 | 20 mg | (3) | 7 |
| low dose | weeks off) | 30 mg | (4) | |
| regimens | 2C (1 week on/3 | 45 mg | (7AML; 1 ALL) | 8 |
| | weeks off) | | | |

At the time of data cut-off, a total of 37 patients (35 AML and 2 ALL) have been treated with HDM201 across the 4 dosing regimens evaluated (refer to Table 1). In regimen 2C, 8 patients (7 AML and 1 ALL) have been treated with HDM201 at the single dose level of 45 mg.

Table 2 provides the characteristics of those patients.

TABLE 2
Patient characteristics

| | Reg 1A | | Reg 1B | | Reg 2A | | Reg 2C | |
|---|---|---|---|---|---|---|---|---|
| | (n = 16) | | (n = 6) | | (n = 7) | | (n = 8) | |
| Median age, years (range) | 70 | (23-81) | 71 | (64-83) | 63 | (26-72) | 75 | (41-81) |
| Male, n (%) | 10 | (63) | 1 | (17) | 7 | (100) | 7 | (88) |
| ECOG PS, n (%) | | | | | | | | |
| 0 | 2 | (13) | 0 | (0) | 4 | (57) | 0 | (0) |
| 1 | 13 | (81) | 5 | (83) | 2 | (29) | 7 | (88) |
| 2 | 1 | (6) | 1 | (17) | 1 | (14) | 1 | (13) |
| Disease history for AML patients | n = 15 | | n = 6 | | n = 7 | | n = 7 | |
| Median prior treatment regimens, | TBC | | TBC | | TBC | | TBC | |
| n (range) | | | | | | | | |
| WHO classification at | | | | | | | | |
| initial diagnosis, n (%) | | | | | | | | |
| AML (BM blasts >30%) | 8 | (53) | 6 | (100) | 6 | (86) | 3 | (43) |
| AML with multilineage dysplasia | 6 | (40) | 0 | (0) | 1 | (14) | 4 | (57) |
| (BM blasts 21-30%) | | | | | | | | |
| Cytogenetics at initial | | | | | | | | |
| diagnosis, n (%) | | | | | | | | |
| Favorable | 0 | (0) | 0 | (0) | 0 | (0) | 0 | (0) |
| Intermediate | 3 | (20) | 2 | (33) | 1 | (14) | 2 | (29) |
| Unfavorable | 3 | (20) | 0 | (0) | 1 | (14) | 2 | (29) |
| Unknown | 3 | (20) | 2 | (33) | 1 | (14) | 0 | (0) |
| Missing | 6 | (40) | 2 | (33) | 4 | (57) | 3 | (43) |
| Prior allogeneic stem cell | 2 | (13) | 1 | (17) | 1 | (14) | 0 | (0) |
| transplant, n (%) | | | | | | | | |

The patient population is further characterized by the following inclusion criteria:
Patient (male or female) ≥18 years of age.
ECOG performance status 0-2.
Relapsed/Refractory AML (both de novo or secondary AML) expect for Acute Promyelocytic Leukemia (APL) with t(15;17), or previously untreated patients who are considered inappropriate candidates for standard induction therapy
In dose escalation only, high and very high risk MDS according to the revised International Prognostic Scoring System (IPSS-R) who have failed prior therapies, such as azacitidine and decitabine (Patients with IPSS-R score of >4.5).
In dose escalation only, relapsed/Refractory Acute Lymphobiastic Leukemia (B-ALL or T-ALL) including Ph+ ALL, or previously untreated patients who are considered inappropriate candidates for standard induction therapy. Patients with Ph+ ALL who show early markers of relapse in MRD surveillance can be considered for inclusion as long as other therapies such as TKIs are exhausted or cannot be given.
Tumor of the patient is TP53 wt characterized by, at a minimum, no mutations in exons 5, 6, 7 and 8, and the p53 status was obtained from a bone-marrow sample, collected no longer than 3 months before signing the main ICF.

Safety and Tolerability Profile of Regimen 2C

In Regimen 2C the most frequent treatment-related AEs (all grades) were thrombocytopenia and anemia (5 patients each, 62.5%), neutropenia and decreased appetite (4 patients each, 50.0%) febrile neutropenia and nausea (3 patients each, 37.5%). Five patients (62.5%) experienced at least one CTCAE grade 3/4 AE related to study drug. The four most frequent CTCAE grade 3/4 AEs related to study drug were: thrombocytopenia (5 patients, 62.5%), anemia, neutropenia and febrile neutropenia (3 patients each, 37.5%).

One DLT was observed in one patient (12.5%) who had tumor lysis syndrome G4 (Table 3).

This clinical study utilized a Bayesian logistic regression model (BLRM) to support dose escalation and estimate the MTD for HDM201. The BLRM enables incorporation of available prior information and updates the model parameters based upon the DLTs observed in the clinical study at varied doses in evaluable patients. Dose selection for the next cohort is based on EWVOC principle which allows only the doses where the probability of exceeding overdose toxicity is less or equal to 25%. The results of the BLRM based on the 1 DLT observed out of 8 evaluable patients in cycle 1 receiving Regimen 2C 45 mg allowed to escalate up to 90 mg.

However, based on the totality of the data observed from all cycles including safety, response rates, PK, PK/PD, the dose of 45 mg for Reg 2C was found most as most preferable for the expansion phase of the clinical study.

TABLE 3
Cycle 1 DLTs in hematological tumors

| | | Dose levels | |
|---|---|---|---|
| | | (number of | |
| | | evaluable | |
| | Dosing Regimen | patients) | DLTs |
| Intermittent | 1A (d 1 Q3W) | 250 mg (3) | 0 |
| high dose | | 350 mg (4) | 0 |
| regimens | | 400 mg (9) | 4 |
| | Total | N = 16 | 4: |
| | | | Infection G3, GVHd |
| | | | reactivation |
| | | | G3, Stomatitis G3 |
| | | | Hypophosphatemia G4 |
| | | | Subarachnoid |
| | | | hemorrhage (fatal) |
| | | | Hypophosphatemia G4 |
| | 1B (d 1, d 8 | 150 mg (6) | 1 |
| | Q4W) | | |
| | Total | N = 6 | 1: |
| | | | Acute Kidney Injury |
| | | | G4 |
| Extended | 2A (2 weeks on/2 | 20 mg (3) | 0 |
| low dose | weeks off) | 30 mg (3) | 0 |
| regimens | Total | N = 6 | 0 |
| | 2C (1 week on/3 | 45 mg (8) | 1 |
| | weeks off) | | |
| | Total | N = 8 | 1 - Tumor Lysis |
| | | | Syndrome G4 |

TABLE 4
All grades and grade 3/4 adverse events, suspected to be study drug
related, by preferred term and regimens - hematological tumors

| | HDM201 | | HDM201 | | HDM201 | | HDM201 | | | |
|---|---|---|---|---|---|---|---|---|---|---|
| | Regimen 1A | | Regimen 1B | | Regimen 2A | | Regimen 2C | | All | |
| | subjects | | subjects | | Subjects | | subjects | | subjects | |
| | N = 16 | | N = 6 | | N = 7 | | N = 8 | | N = 37 | |
| MEDDRA | All | Grade | All | Grade | All | Grade | All | Grade | All | Grade |
| Preferred | Grades | 3/4 | Grades | 3/4 | Grades | 3/4 | Grades | 3/4 | Grades | 3/4 |
| Term | n (%) | n (%) | n (%) | n (%) | n (%) | n (%) | n (%) | n (%) | n (%) | n (%) |
| Total | 15 (93.8) | 12 (75.0) | 5 (83.3) | 5 (83.3) | 5 (71.4) | 3 (42.9) | 8 (100) | 5 (62.5) | 33 (89.2) | 25 (67.6) |
| Nausea | 10 (62.5) | 0 | 4 (66.7) | 0 | 3 (42.9) | 0 | 3 (37.5) | 0 | 20 (54.1) | 0 |
| Thrombocytopenia/ | 8 (50.0) | 8 (50.0) | 3 (50.0) | 3 (50.0) | 2 (28.6) | 2 (28.6) | 5 (62.5) | 5 (62.5) | 18 (48.6) | 18 (48.6) |
| Platelet | | | | | | | | | | |
| Count | | | | | | | | | | |
| Decreased | | | | | | | | | | |
| Anaemia | 6 (37.5) | 4 (25.0) | 2 (33.3) | 2 (33.3) | 3 (42.9) | 2 (28.6) | 5 (62.5) | 3 (37.5) | 16 (43.2) | 11 (29.7) |
| Neutropenia/ | 6 (37.5) | 6 (37.5) | 1 (16.7) | 1 (16.7) | 1 (14.3) | 1 (14.3) | 4 (50.0) | 3 (37.5) | 12 (32.4) | 11 (29.7) |
| Neutrophil | | | | | | | | | | |
| Count | | | | | | | | | | |
| Decreased | | | | | | | | | | |
| Febrile | 4 (25.0) | 4 (25.0) | 2 (33.3) | 2 (33.3) | 2 (28.6) | 2 (28.6) | 3 (37.5) | 3 (37.5) | 11 (29.7) | 11 (29.7) |
| Neutropenia | | | | | | | | | | |
| Decreased | 4 (25.0) | 0 | 1 (16.7) | 0 | 1 (14.3) | 0 | 4 (50.0) | 0 | 10 (27.0) | 0 |
| Appetite | | | | | | | | | | |
| Tumour Lysis | 7 (43.8) | 7 (43.8) | 0 | 0 | 1 (14.3) | 1 (14.3) | 1 (12.5) | 1 (12.5) | 9 (24.3) | 9 (24.3) |
| Syndrome | | | | | | | | | | |
| Vomiting | 5 (31.3) | 0 | 0 | 0 | 1 (14.3) | 0 | 1 (12.5) | 0 | 7 (18.9) | 0 |
| Diarrhoea | 4 (25.0) | 0 | 0 | 0 | 0 | 0 | 2 (25.0) | 0 | 6 (16.2) | 0 |
| Fatigue | 4 (25.0) | 0 | 0 | 0 | 0 | 0 | 2 (25.0) | 0 | 6 (16.2) | 0 |
| Asthenia | 2 (12.5) | 0 | 1 (16.7) | 0 | 0 | 0 | 2 (25.0) | 0 | 5 (13.5) | 0 |
| Pyrexia | 3 (18.8) | 0 | 0 | 0 | 1 (14.3) | 0 | 0 | 0 | 4 (10.8) | 0 |
| White Blood | 1 (6.3) | 0 | 0 | 0 | 1 (14.3) | 1 (14.3) | 2 (25.0) | 2 (25.0) | 4 (10.8) | 3 (8.1) |
| Cell Count | | | | | | | | | | |
| Decreased | | | | | | | | | | |
| Amylase | 3 (18.8) | 0 | 0 | 0 | 0 | 0 | 0 | 0 | 3 (8.1) | 0 |
| Increased | | | | | | | | | | |
| Lipase | 3 (18.8) | 2 (12.5) | 0 | 0 | 0 | 0 | 0 | 0 | 3 (8.1) | 2 (5.4) |
| Increased | | | | | | | | | | |
| Acute Kidney | 1 (6.3) | 0 | 1 (16.7) | 1 (16.7) | 1 (14.3) | 0 | | | 3 (8.1) | 1 (2.7) |
| Injury | | | | | | | | | | |
| Blood | 1 (6.3) | 0 | 0 | 0 | 1 (14.3) | 0 | 1 (12.5) | 1 (12.5) | 3 (8.1) | 1 (2.7) |
| Creatinine | | | | | | | | | | |
| Increased | | | | | | | | | | |
| Blood | 1 (6.3) | 0 | 1 (16.7) | 0 | 0 | 0 | 0 | 0 | 3 (8.1) | 0 |
| Phosphorus | | | | | | | | | | |
| Increased | | | | | | | | | | |
| Hyperbilirubinaemia | 1 (6.3) | 0 | 1 (16.7) | 0 | 1 (14.3) | 0 | 0 | 0 | 3 (8.1) | 0 |
| Hyperphosphataemia | 1 (6.3) | 0 | 1 (16.7) | 0 | 0 | 0 | 1 (12.5) | 0 | 3 (8.1) | 0 |
| Abdominal | 2 (12.5) | 0 | 0 | 0 | 0 | 0 | 0 | 0 | 2 (5.4) | 0 |
| Pain | | | | | | | | | | |
| Atrial | 2 (12.5) | 0 | 0 | 0 | 0 | 0 | 0 | 0 | 2 (5.4) | 0 |
| Fibrillation | | | | | | | | | | |
| Blood Bilirubin | 1 (6.3) | 0 | 1 (16.7) | 0 | 0 | 0 | 0 | 0 | 2 (5.4) | 0 |
| Increased | | | | | | | | | | |
| C-Reactive | 1 (6.3) | 0 | 0 | 0 | 1 (14.3) | 1 (14.3) | 0 | 0 | 2 (5.4) | 1 (2.7) |
| Protein | | | | | | | | | | |
| Increased | | | | | | | | | | |
| Constipation | 0 | 0 | 1 (16.7) | 0 | | | 1 (12.5) | 0 | 2 (5.4) | 0 |
| Dyspepsia | 1 (6.3) | 0 | 0 | 0 | 1 (14.3) | 0 | 0 | 0 | 2 (5.4) | 0 |
| Gamma- | 2 (12.5) | 1 (6.3) | 0 | 0 | 0 | 0 | 0 | 0 | 2 (5.4) | 1 (2.7) |
| Glutamyltransferase | | | | | | | | | | |
| Increased | | | | | | | | | | |
| General | 2 (12.5) | 0 | 0 | 0 | 0 | 0 | 0 | 0 | 2 (5.4) | 0 |
| Physical | | | | | | | | | | |
| Health | | | | | | | | | | |
| Deterioration | | | | | | | | | | |
| Gingival | 2 (12.5) | 0 | 0 | 0 | 0 | 0 | 0 | 0 | 2 (5.4) | 0 |
| Bleeding | | | | | | | | | | |
| Headache | 1 (6.3) | 0 | 1 (16.7) | 0 | 0 | 0 | 0 | 0 | 2 (5.4) | 0 |
| Hyperuricaemia | 2 (12.5) | 1 (6.3) | 2 (33.3) | 0 | 1 (14.3) | 0 | | | 2 (5.4) | 1 (2.7) |
| Hypocalcaemia | 2 (12.5) | 0 | 0 | 0 | 0 | 0 | 0 | 0 | 2 (5.4) | 0 |
| Hypophosphataemia | 2 (12.5) | 2 (12.5) | 0 | 0 | 0 | 0 | 0 | 0 | 2 (5.4) | 2 (5.4) |
| Hyponatraemia | 1 (6.3) | 0 | 1 (16.7) | 0 | 0 | 0 | 0 | 0 | 2 (5.4) | 0 |
| Leukopenia | 2 (12.5) | 2 (12.5) | 0 | 0 | 0 | 0 | 0 | 0 | 2 (5.4) | 2 (5.4) |
| Malaise | 1 (6.3) | 0 | 0 | 0 | 1 (14.3) | 0 | 0 | 0 | 2 (5.4) | 0 |
| Mouth | 2 (12.5) | 0 | 0 | 0 | 0 | 0 | 0 | 0 | 2 (5.4) | 0 |
| Haemorrhage | | | | | | | | | | |
| Pancytopenia | 0 | 0 | 1 (16.7) | 1 (16.7) | 0 | 0 | 1 (12.5) | 1 (12.5) | 2 (5.4) | 2 (5.4) |
| Stomatitis | 1 (6.3) | 1 (6.3) | 0 | 0 | 0 | 0 | 1 (12.5) | 0 | 2 (5.4) | 1 (2.7) |
| Abdominal | 1 (6.3) | 0 | 0 | 0 | 0 | 0 | 0 | 0 | 1 (2.7) | 0 |
| Discomfort | | | | | | | | | | |
| Abdominal | 1 (6.3) | 0 | 0 | 0 | 0 | 0 | 0 | 0 | 1 (2.7) | 0 |
| Pain Upper | | | | | | | | | | |
| Arthralgia | 1 (6.3) | 0 | 0 | 0 | 0 | 0 | 0 | 0 | 1 (2.7) | 0 |
| Atypical | 1 (6.3) | 0 | 0 | 0 | 0 | 0 | 0 | 0 | 1 (2.7) | 0 |
| Pneumonia | | | | | | | | | | |
| Balanitis | 1 (6.3) | 0 | 0 | 0 | 0 | 0 | 0 | 0 | 1 (2.7) | 0 |
| Candida | | | | | | | | | | |
| Blood Alkaline | 1 (6.3) | 0 | 0 | 0 | 0 | 0 | 0 | 0 | 1 (2.7) | 0 |
| Phosphatase | | | | | | | | | | |
| Increased | | | | | | | | | | |
| Blood Uric | 1 (6.3) | 1 (6.3) | 0 | 0 | 0 | 0 | 0 | 0 | 1 (2.7) | 1 (2.7) |
| Acid | | | | | | | | | | |
| Increased | | | | | | | | | | |
| Bone Marrow | 0 | 0 | 0 | 0 | 1 (14.3) | 1 (14.3) | 0 | 0 | 1 (2.7) | 1 (2.7) |
| Failure | | | | | | | | | | |
| Cardiac | 1 (6.3) | 0 | 0 | 0 | 0 | 0 | 0 | 0 | 1 (2.7) | 0 |
| Failure | | | | | | | | | | |
| Cellulitis | 0 | 0 | 1 (16.7) | 1 (16.7) | 0 | 0 | 0 | 0 | 1 (2.7) | 1 (2.7) |
| Chronic Graft | 1 (6.3) | 1 (6.3) | 0 | 0 | 0 | 0 | 0 | 0 | 1 (2.7) | 1 (2.7) |
| Versus Host | | | | | | | | | | |
| Disease | | | | | | | | | | |
| Conjunctival | 1 (6.3) | 0 | 0 | 0 | 0 | 0 | 0 | 0 | 1 (2.7) | 0 |
| Haemorrhage | | | | | | | | | | |
| Cough | 1 (6.3) | 0 | 0 | 0 | 0 | 0 | 0 | 0 | 1 (2.7) | 0 |
| Cystitis Viral | 1 (6.3) | 0 | 0 | 0 | 0 | 0 | 0 | 0 | 1 (2.7) | 0 |
| Cytomegalovirus | 0 | 0 | 0 | 0 | 1 (14.3) | 0 | 0 | 0 | 1 (2.7) | 0 |
| Infection | | | | | | | | | | |
| Dyspnoea | 1 (6.3) | 1 (6.3) | 0 | 0 | 0 | 0 | 0 | 0 | 1 (2.7) | 1 (2.7) |
| Epistaxis | 0 | 0 | 0 | 0 | 0 | 0 | 1 (12.5) | 0 | 1 (2.7) | 0 |
| Graft Versus | 1 (6.3) | 0 | 0 | 0 | 0 | 0 | 0 | 0 | 1 (2.7) | 0 |
| Host Disease | | | | | | | | | | |
| Haemoglobin | 1 (6.3) | 0 | 0 | 0 | 0 | 0 | 0 | 0 | 1 (2.7) | 0 |
| Decreased | | | | | | | | | | |
| Hyperglycaemia | 1 (6.3) | 0 | 0 | 0 | 0 | 0 | 0 | 0 | 1 (2.7) | 0 |
| Hyperkalaemia | 0 | 0 | 1 (16.7) | 1 (16.7) | 0 | 0 | 0 | 0 | 1 (2.7) | 1 (2.7) |
| Hypoalbuminaemia | 1 (6.3) | 0 | 0 | 0 | 0 | 0 | 0 | 0 | 1 (2.7) | 0 |
| Hypomagnesaemia | 1 (6.3) | 0 | 0 | 0 | 0 | 0 | 0 | 0 | 1 (2.7) | 0 |
| Hypokalaemia | 0 | 0 | 1 (16.7) | 0 | 0 | 0 | 0 | 0 | 1 (2.7) | 0 |
| Hypotension | 1 (6.3) | 0 | 0 | 0 | 0 | 0 | 0 | 0 | 1 (2.7) | 0 |
| Infection | 1 (6.3) | 1 (6.3) | 0 | 0 | 0 | 0 | 0 | 0 | 1 (2.7) | 1 (2.7) |
| Infective | 0 | 0 | 0 | 0 | 0 | 0 | 1 (12.5) | 0 | 1 (2.7) | 0 |
| Glossitis | | | | | | | | | | |
| Influenza Like | 1 (6.3) | 0 | 0 | 0 | 0 | 0 | 0 | 0 | 1 (2.7) | 0 |
| Illness | | | | | | | | | | |
| Iron Overload | 1 (6.3) | 0 | 0 | 0 | 0 | 0 | 0 | 0 | 1 (2.7) | 0 |
| Lip Oedema | 1 (6.3) | 0 | 0 | 0 | 0 | 0 | 0 | 0 | 1 (2.7) | 0 |
| Macular | 0 | 0 | 1 (16.7) | 1 (16.7) | 0 | 0 | 0 | 0 | 1 (2.7) | 1 (2.7) |
| Oedema | | | | | | | | | | |
| Oedema | 1 (6.3) | 0 | 0 | 0 | 0 | 0 | 0 | 0 | 1 (2.7) | 0 |
| Oedema | | | 1 (16.7) | 0 | 0 | 0 | 0 | 0 | 1 (2.7) | 0 |
| Peripheral | | | | | | | | | | |
| Oral Disorder | 1 (6.3) | 0 | 0 | 0 | 0 | 0 | 0 | 0 | 1 (2.7) | 0 |
| Pain In | 1 (6.3) | 0 | 0 | 0 | 0 | 0 | 0 | 0 | 1 (2.7) | 0 |
| Extremity | | | | | | | | | | |
| Pneumonia | 1 (6.3) | 0 | 0 | 0 | 0 | 0 | 0 | 0 | 1 (2.7) | 0 |
| Pollakiuria | 1 (6.3) | 0 | 0 | 0 | 0 | 0 | 0 | 0 | 1 (2.7) | 0 |
| Rash | 1 (6.3) | 0 | 0 | 0 | 0 | 0 | 0 | 0 | 1 (2.7) | 0 |
| Rash | 1 (6.3) | 0 | 0 | 0 | 0 | 0 | 0 | 0 | 1 (2.7) | 0 |
| Erythematous | | | | | | | | | | |
| Rash Papular | 1 (6.3) | 0 | 0 | 0 | 0 | 0 | 0 | 0 | 1 (2.7) | 0 |
| Rectal | 0 | 0 | 1 (16.7) | 0 | 0 | 0 | 0 | 0 | 1 (2.7) | 0 |
| Haemorrhage | | | | | | | | | | |
| Renal Failure | 0 | 0 | 0 | 0 | 0 | 0 | 1 (12.5) | 1 (12.5) | 1 (2.7) | 1 (2.7) |
| Skin | 1 (6.3) | 0 | 0 | 0 | 0 | 0 | 0 | 0 | 1 (2.7) | 0 |
| Exfoliation | | | | | | | | | | |
| Subarachnoid | 1 (6.3) | 1 (6.3) | 0 | 0 | 0 | 0 | 0 | 0 | 1 (2.7) | 1 (2.7) |
| Haemorrhage | | | | | | | | | | |
| Weight | 1 (6.3) | 0 | 0 | 0 | 0 | 0 | 0 | 0 | 1 (2.7) | 0 |
| Decreased | | | | | | | | | | |

Preferred terms are sorted in descending frequency of <all grades> column, as reported in the <All subjects> column.
A subject with multiple occurrences of an AE under one treatment is counted only once in the AE category For that treatment.
A subject with multiple adverse events is counted only once in the total row.
Only AEs occurring during treatment or within 30 days of the last study medication are reported.

Updated safety data for the dosing regimens 1A (at 250 mg daily dose), 1B (at 120 mg daily dose) and 2C (at 45 mg daily dose) with an cut-off date of 15 Jan. 2018 are provided in the Table 4A.

TABLE 4A
All grades and grade 3/4 adverse events (≥10%), suspected
to be study drug related, in patients with hematological tumors.

HDM201 1A
250 mg
N = 10

| | | |
|---|---|---|
| | All Grades | Grade 3/4 |
| Grouping term | n (%) | n (%) |

Hematological toxicity

| | | |
|---|---|---|
| Thrombocytopenia | 5 (50.0) | 5 (50.0) |
| Anemia | 5 (50.0) | 3 (30.0) |
| Neutropenia | 4 (40.0) | 3 (30.0) |
| Febrile Neutropenia | 2 (20.0) | 2 (20.0) |
| Leukopenia | 1 (10.0) | 1 (10.0) |

GI toxicity

| | | |
|---|---|---|
| Nausea | 3 (30.0) | 0 |
| Diarrhoea | 3 (30.0) | 0 |

Metabolism and nutrition disorders

| | | |
|---|---|---|
| Hypocalcaemia | 3 (30.0) | 1 (10.0) |
| Blood Uric Acid Increased | 2 (20.0) | 1 (10.0) |
| Hyperkalaemia | 2 (20.0) | 1 (10.0) |
| Hypomagnesaemia | 2 (20.0) | 0 |
| Hyperuricaemia | 1 (10.0) | 1 (10.0) |

Investigations

| | | |
|---|---|---|
| Blood Creatinine Increased | 2 (20.0) | 0 |
| Amylase Increased | 1 (10.0) | 1 (10.0) |

Other

| | | |
|---|---|---|
| Tumour Lysis Syndrome | 5 (50.0) | 5 (50.0) |
| Aspiration | 1 (10.0) | 1 (10.0) |
| Megakaryocytes Abnormal | 1 (10.0) | 1 (10.0) |
| Vomiting, Blood Bilirubin | 1 (10.0) | 0 |
| Increased, Blood Phosphorus | | |
| Increased, | | |
| Hyperphosphataemia, Fatigue, | | |
| Abdominal Pain, Alopecia, | | |
| Asthenia, Dizziness, Periorbital | | |
| Haematoma, Renal Failure | | |

HDM201 1B
120 mg
N = 23

| | |
|---|---|
| All Grades | Grade 3/4 |
| n (%) | n (%) |

Hematological toxicity

| | | |
|---|---|---|
| Thrombocytopenia | 10 (43.5) | 10 (43.5) |
| Anemia | 7 (30.4) | 2 (8.7) |
| Neutropenia | 4 (17.4) | 4 (17.4) |
| Febrile Bone Marrow Aplasia | 3 (13.0) | 2 (8.7) |
| Leukopenia | 3 (13.0) | 3 (13.0) |

GI toxicity

| | | |
|---|---|---|
| Nausea | 6 (26.1) | 0 |
| Vomiting | 3 (13.0) | 0 |

Other

| | | |
|---|---|---|
| Asthenia | 3 (13 0) | 1 (4.3) |
| Tumour Lysis Syndrome | 4 (17.4) | 3 (13.0) |

HDM201 2C
45 mg
N = 25

| | |
|---|---|
| All Grades | Grade 3/4 |
| n (%) | n (%) |

Hematological toxicity

| | | |
|---|---|---|
| Anemia | 13 (52.0) | 9 (36.0) |
| Thrombocytopenia | 13 (52.0) | 12 (48.0) |
| Neutropenia | 11 (44.0) | 10 (40.0) |
| Febrile Neutropenia | 8 (32.0) | 8 (32.0) |
| Leukopenia | 5 (20.0) | 5 (20.0) |

GI toxicity

| | | |
|---|---|---|
| Nausea | 12 (48.0) | 0 |
| Decreased Appetite | 6 (24.0) | 0 |
| Vomiting | 6 (24.0) | 0 |

Other

| | | |
|---|---|---|
| Fatigue | 5 (20.0) | 2 (8.0) |
| Alopecia | 3 (12.0) | 0 |
| Tumour Lysis Syndrome | 3 (12.0) | 3 (12.0) |

Anti-Tumor Activity/Efficacy of Regimen 2C

Of 8 patients treated at the 45 mg dose in regimen 2C, 7 patients had at least one post-baseline efficacy assessment and the overall response rate was 57.1% (see Table 5). One patient achieved complete response which lasted 152 days and three patients achieved CRi (morphologic CR with incomplete blood count recovery) which lasted 40 days for one of them. For another patient the CRi was ongoing at the time of the data cut off (see Table 6). Compared with the other high dose intermittent (1A and 1B) and the other extended low dose (2A) regimens regimen 2C appears to be clinically most effective.

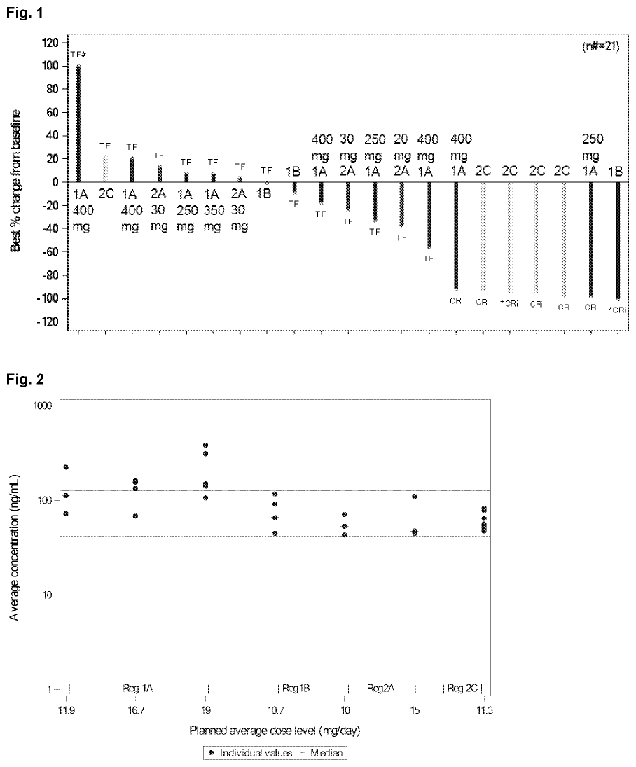

Regimen 2C also shows strong efficacy with respect to the best percentage change in blast percentage in bone marrow (BM) aspirate in AML patients (patients with available bone marrow aspirate), see FIG. 1.

Considering that doses up to 30 mg were evaluated in regimen 2A (2 weeks on/2 week off) without evidence of clinically meaningful activity, it appears that a dose much lower than 45 mg in regimen 2C (1 week on/3 weeks off) is less preferred with respect to efficacy.

Although no hematologic toxicity meeting DLT criteria was observed at the 45 mg dose, the patient with CR had a count recovery time of 32 days and the 3 other patients had CR with incomplete count recovery (see Table 6). Given the increased risk of prolonged count recovery times doses much higher than 45 mg in regimen 2C appear to be less preferred with respect to tolerability.

TABLE 5
Anti-tumor activity

| | BOR, n (%) | | | | |
|---|---|---|---|---|---|
| | Regimen | Regimen | Regimen | Regimen | |
| | 1A | 1B | 2A | 2C | Total |
| | (n = 15) | (n = 6) | (n = 7) | (n = 7) | (N = 35) |
| CR | 2 (13) | 0 (0) | 0 (0) | 1 (14) | 3 (9) |
| CRi | 0 (0) | 1 (17) | 0 (0) | 3 (43) | 4 (11) |
| ORR (CR + | 2 (13) | 1 (17) | 0 (0) | 4 (57) | 7 (20) |
| CRi + PR) | | | | | |

BOR, best overall response;
CR, complete response;
CRi, morphologic CR with incomplete blood count recovery;
ORR, overall response rate

TABLE 6
Characteristics of AML patients with CR/CRi

| | | | | | | |
|---|---|---|---|---|---|---|
| | | | | Time to | Count recovery | Duration of |
| | | Age | Cytogenetics at | CR/CRi | time from C1D1 | CR/CRi |
| Regimen | Dose | (years) | diagnosis | (weeks) | (days) | (days) |

CR patients

| | | | | | | | |
|---|---|---|---|---|---|---|---|
| High- | 250 | mg | 81 | Intermediate | 7 | 48 | 50 |
| dose: 1A | | | | | | | |
| High- | 400 | mg | 71 | Intermediate | 6 | 40 | 23 |
| dose: 1A | | | | | | | |
| Low- | 45 | mg | 75 | Missing | 5 | 32 | 152 |
| dose: 2C | | | | | | | |

CRi patients

| | | | | | | | |
|---|---|---|---|---|---|---|---|
| High- | 150 | mg | 83 | Intermediate | 11 | NA | 51 |
| dose: 1B | | | | | | | |
| Low- | 45 | mg | 80 | Missing | 7 | NA | 40 |
| dose: 2C | | | | | | | |
| Low- | 45 | mg | 70 | Unfavorable | 12 | NA | 1* |
| dose: 2C | | | | | | | |
| Low- | 45 | mg | 79 | Intermediate | 4 | NA | 1 ^{†} |
| dose: 2C | | | | | | | |

*Patient withdrew consent while on CRi
^{†} Patient ongoing at the time of data cut off

Updated efficacy data for the dosing regimens IA (at 250 mg daily dose), 1B (at 120 mg daily dose) and 2C (at 45 mg daily dose) for patients with hematological tumors with a cut-off date of 15 Jan. 2018 are provided in the FIGS. 3-5 and Table 7.

TABLE 7
Updated anti-tumor activity (cut-off: 15 Jan. 2018)

| | Regimen 1A | | Regimen 1B | | Regimen 2C | |
|---|---|---|---|---|---|---|
| Patients (n) | 10 | | 23 | | 25 | |
| CR | 1 | | 1 | | 2 | |
| CRi | 1 | | 0 | | 4 | |
| Time to CR/CRi | 24-50 | days | 30 | days | 34-49 | days |
| Duration of | 50-125 | days | 1 day (DoR | | 1 (DoR | |
| CR/CRi | | | censored) | | censored)-152 days | |
| Mean duration | 9.1 | weeks | 8.7 | weeks | 12.1 | weeks |
| of exposure | | | | | | |

Clinical PK

Pharmacokinetic data have been evaluated throughout the course of the clinical study. Non-compartmental PK analysis showed a median time to reach maximum plasma concentrations ranging from 2.0 to 5.8 h across the dose range (2 to 350 mg). A preliminary dose proportionality assessment showed approximately dose proportional PK (AUClast and Cmax) over the dose range studied. For the majority of dose cohorts, the inter-patient variability (CV % Geo-mean) for AUClast and Cmax was low to moderate (6 to 58.5%). Furthermore, an integrated analysis of all available HDM201 concentrations was conducted using a population approach. The PK of HDM201 was best described by a 1-compartment PK model with a delayed zero- and first-order absorption process, and a linear clearance. Body weight was identified as a statistically significant covariate on apparent central volume of distribution (Vc/F), in which Vc/F increased with increasing body weight.

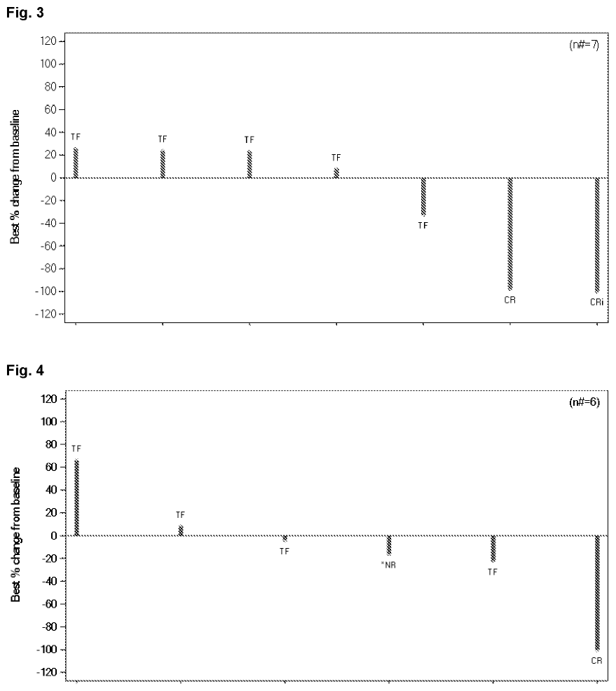

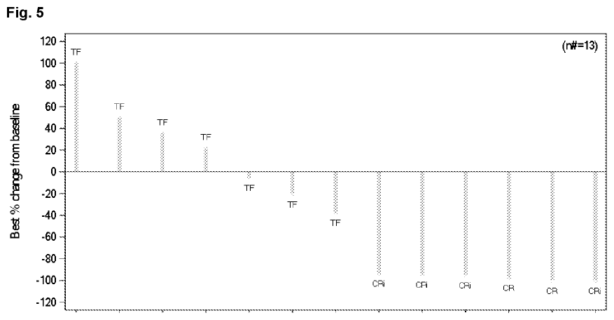

To further support the 45 mg for HDM201, compartmental PK modeling was used to estimate the individual average concentration during cycle 1 for patients with hematological tumors treated at 45 mg on regimen 2C (FIG. 2). For all patients with measured PK, the estimated average drug concentrations during cycle 1 were above the most conservative average tumor stasis concentration of about 41 ng/mL per cycle determined from PKPD modeling of preclinical data (human SJSA-1 xenograft rat model).

### Example 2: Drug Product

The drug product consists of HDM201 succinic acid drug substance filled directly into hard gelatin capsules (HGC), and does not contain any other excipients. The drug product is provided in four dosage strengths: 1 mg, 2.5 mg, 10 mg and 100 mg (based on the weight of the free form), intended for oral use. The 1 mg strength capsule is a “Size 3” yellow HGC, the 2.5 mg strength capsule is a “Size 3” Swedish Orange HGC, the 10 mg strength capsule is a “Size 1” Grey HGC, and the 100 mg is a “Size 0” Swedish Orange HGC. The drug product is packaged in child resistant, induction sealed High Density Polyethylene (HDPE) bottles.

1. The HDM2-p53 interaction inhibitor drug (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (HDM201) or a pharmaceutically acceptable non-covalent derivative (including salt, solvate, hydrate, complex, co-crystal) thereof
for use in the treatment of hematological tumors,
wherein the drug is administered on each of the first 6 to 8 days of a 28 days treatment cycle,
wherein the treatment is composed of at least two 28 days treatment cycles, and
wherein the daily drug dose is from 40 mg to 90 mg.

2. The HDM2-p53 interaction inhibitor drug HDM201, or non-covalent derivative thereof, for use in the treatment of hematological tumors according to claim 1, wherein the daily drug dose is from 40 mg to 60 mg.

3. The HDM2-p53 interaction inhibitor drug HDM201, or non-covalent derivative thereof, for use in the treatment of hematological tumors according to claim 1, wherein the daily drug dose is from 40 mg to 50 mg.

4. The HDM2-p53 interaction inhibitor drug HDM201, or non-covalent derivative thereof, for use in the treatment of hematological tumors according to claim 1, wherein daily drug dose is 45 mg.

5. The HDM2-p53 interaction inhibitor drug HDM201, or non-covalent derivative thereof, for use in the treatment of hematological tumors according to claim 1, wherein the drug is administered once daily on each of the first 7 days (first week) of a 28 days (4 weeks) treatment cycle and the daily drug dose is 45 mg.

6. The HDM2-p53 interaction inhibitor drug HDM201, or non-covalent derivative thereof, for use in the treatment of hematological tumors according to claim 1, wherein the drug is present as co-crystal, preferably present as succinic acid co-crystal.

7. The HDM2-p53 interaction inhibitor drug HDM201, or non-covalent derivative thereof, for use in the treatment of hematological tumors according to claim 1, wherein the drug is present as solvate, preferably present as hydrate.

8. The HDM2-p53 Interaction inhibitor drug HDM201, or non-covalent derivative thereof, for use in the treatment of hematological tumors according to claim 1, wherein the drug is present as non-covalent derivative, preferably present as non-covalent derivative comprising succinic acid or water, more preferably present as non-covalent derivative comprising succinic acid.

9. The HDM2-p53 interaction inhibitor drug HDM201, or non-covalent derivative thereof, for use in the treatment of hematological tumors according to claim 1, wherein the hematological tumor is a leukemia.

10. The HDM2-p53 Interaction Inhibitor drug HDM201, or non-covalent derivative thereof, for use in the treatment of hematological tumors according to claim 1, wherein the hematological tumor is selected from acute myeloid leukemia (AML), myelodysplastic syndrome (MDS), and acute lymphoblastic leukemia (ALL).

11. The HDM2-p53 Interaction inhibitor drug HDM201, or non-covalent derivative thereof, for use in the treatment of hematological tumors according to claim 1, wherein the hematological tumor is a TP53 wild-type hematological tumor.

12. The HDM2-p53 interaction Inhibitor drug HDM201, or non-covalent derivative thereof, for use in the treatment of hematological tumors according to claim 1, wherein the hematological tumor is a relapsed/refractory hematological tumor.

13. HDM2-p53 Interaction inhibitor drug HDM201, or non-covalent derivative thereof, for use in the treatment of hematological tumors according to claim 1, wherein the hematological tumor is a relapsed refractory TP53 wild-type hematological tumor selected from acute myeloid leukemia (AMI), myelodysplastic syndrome (MDS), and acute lymphoblastic leukemia (ALL).

14. The HDM2-p53 Interaction inhibitor drug (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (HDM201) as succinic acid co-crystal for use in the treatment of relapsed/refractory TP53 wild-type hematological tumors selected from acute myeloid leukemia (AML), myelodysplastic syndrome (MDS), and acute lymphoblastic leukemia (ALL),
wherein the drug is administered on each of the first 7 days of a 28 days treatment cycle,
wherein the treatment is composed of at least two 28 days treatment cycles, and
wherein the daily drug dose is 45 mg.

15. The HDM2-p53 interaction Inhibitor drug (S)-5-(5-Chloro-1-methyl-2-oxo-1,2-dihydro-pyridin-3-yl)-6-(4-chloro-phenyl)-2-(2,4-dimethoxy-pyrimidin-5-y)-1-isopropyl-5,6-dihydro-1H-pyrrolo[3,4-d]imidazol-4-one (HDM201) as succinic acid co-crystal for use in the treatment of relapsed/refractory TP53 wild-type acute myeloid leukemia (AML),
wherein the drug is administered on each of the first 7 days of a 28 days treatment cycle,
wherein the treatment is composed of at least two 28 days treatment cycles, and
wherein the daily drug dose is 45 mg.

16. The HDM2-p53 Interaction inhibitor drug HDM201, or non-covalent derivative thereof, for use in the treatment of hematological tumors according to claim 1, wherein the HDM201 drug is combined with one or more other anti-cancer agents, preferably said anti-cancer agent(s) is(are) selected from: FLT3 inhibitors (e.g. gilterinib, quizartinib, midostaurin), BCL2 inhibitors (e.g. navitoclax, venetoclax), other HDM2 inhibitors (e.g. idasanutlin, AMG232, DS-3032B, ALRN6924/ATSP7041), hypomethylating agents (HMA) (e.g. Vidaza [azacytidine, 5-azacytidine], Dacogen [decitabine], guadecitabine), anthracyclines (e.g. idarubicin, daunorubicin, doxorubicin, epirubicin); anti-CD33 antibodies (e.g. Mylotarg [gemtuzumab], vadastuximab) and other agents (e.g. AraC [cytarabine, aracytine]).

17. The HDM2-p53 Interaction inhibitor drug HDM201, or non-covalent derivative thereof, for use in the treatment of hematological tumors according to claim 1, wherein the drug HDM201 is combined with one or more other therapeutical active agents selected from midostaurin, azacytidine, and cytarabine.
